(19) United States
(12) Patent Application Publication
(10) Pub. No.: US 2008/0173314 A1
(43) Pub. Date: Jul. 24, 2008

(54) DrapeCaddy

(76) Inventor: Steve J. Brown, Austin, TX (US)

Correspondence Address: WILSON DANIEL SWAYZE, JR., 3804 CLEARWATER CT., PLANO, TX, 75025, US

(21) Appl. No.: 11/625,292

(22) Filed: Jan. 20, 2007

(51) Int. Cl. A61B 19/08

(52) U.S. Cl. 128/849

(57) ABSTRACT

A surgical drape support apparatus includes a base section, a side section coupled to the base section, a curved section coupled to the side section, a top section coupled to the curved section. The side section, the curved section and the top section include a reduced thickness portion. The side section or section and the top section include an inward extending projection. The base section includes a first raised rib projection and a second raised rib projection substantially perpendicular to the first raised rib projection.

### FIELD OF THE INVENTION

This invention relates to an apparatus for supporting a surgical drape and more particularly relates to an apparatus for supporting a drape during surgery.

### BACKGROUND

Drapes sometimes are used during surgery to cover the area surrounding that portion of the body undergoing surgery. This is done to reduce the possibility of contamination to the area in which surgery is being performed from the adjacent area. For example, a plastic sheet may be used with an opening at the area of the surgery. The edges of the opening may be taped to the patient's body to hold it tightly in place.

U.S. Pat. No. 4,223,669 discloses a surgical drape support apparatus including a generally U-shaped horizontal base frame having a longitudinal section, transverse sections extending from the longitudinal section adjacent to the ends thereof substantially perpendicular to the longitudinal section, a support section extending generally vertically from the longitudinal section adjacent one of the transverse sections, an upper section extending from the support section adjacent the free end thereof, the upper section extending generally horizontally from the support section substantially parallel to and above the transverse section closest thereto, the upper section and the support section having passages therethrough and the upper section and the support section having a plurality of spaced openings along at least a portion of their lengths with the openings being oriented toward the area circumscribed by the base frame.

### SUMMARY OF THE INVENTION

The present invention provides a surgical drape support apparatus which is called the DrapeCaddy and which significantly reduces a patient's discomfort. Also, the DrapeCaddy of the invention reduces a patient's anxieties.

The DrapeCaddy may be used for “visualization of the patient during conscious sedation efforts and to maintain a sterile field during the procedure” and may be used for procedures of the upper chest region such as Permanent Pacemaker and Implantable Cardiac Defibrillator implants and other associated procedures requiring vascular access to the subclavian vessels.

The DrapeCaddy has been used as an anesthesia screen for procedures where traditionally standard IV poles would be used as drape holders, or drape stands.

Much like the traditional “anesthesia screens” requiring IV poles, the sterile drape is secured to the DrapeCaddy using clips which secure the drape to the device thus helping to maintain sterile integrity of the operative field distal to the position of the DrapeCaddy.

The device is extremely beneficial in those procedures where conscious sedation and short-term general anesthesia is used. Under these circumstances, it is important that the staff members visualize the patient during these sedation periods. It is most beneficial for those procedures requiring TIVA, or Total Intravenous Anesthesia.

The DrapeCaddy of the present invention is simple in design and convenient to use. Also, the surgical DrapeCaddy of the invention is mold injected ABS material in a TS16949 certified US manufacturing facility.

A surgical drape support apparatus includes a base section, a side section coupled to the base section, a curved section coupled to the side section, a top section coupled to the curved section.

The side section, the curved section and the top section include a reduced thickness portion.

The side section or section and the top section include an inward extending projection.

The base section includes a first raised rib projection, a second raised rib projection substantially perpendicular to the first raised rib projection and a third raised rib projection which extends across the base section.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a cross-sectional view of a side view of the DrapeCaddy of the present invention;

FIG. 2 illustrates a bottom view of the base section of the DrapeCaddy of the present invention;

FIG. 3 illustrates a side view of the DrapeCaddy of the present invention;

FIG. 4 illustrates a perspective view of the DrapeCaddy of the present invention;

FIG. 5 illustrates a cross-sectional view of the DrapeCaddy of the present invention;

FIG. 6 illustrates a cross section of a portion of the DrapeCaddy of the present invention;

FIG. 7 illustrates a cross section of another portion of the DrapeCaddy of the present invention;

### DETAILED DESCRIPTION

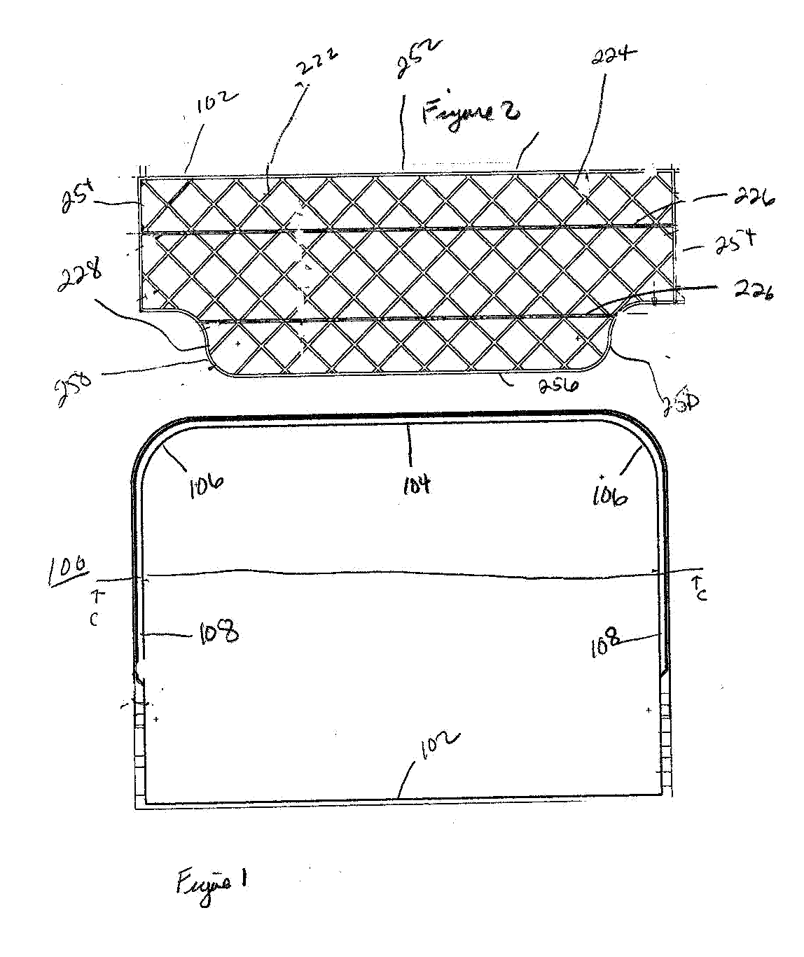

FIG. 1 illustrates a side view of the DrapeCaddy shown generally as 100. The DrapeCaddy 100 which is a rigid support is used to support a surgical cover over a patient. The DrapeCaddy 100 includes a base section 102 to be positioned under the head, shoulders, or upper torso of the patient and a top section 104 to support the surgical cover (not shown). The DrapeCaddy 100 additionally includes a side section 108 in the longitudinal direction between the top section 104 which is in the traverse direction and the base section 102 which is in the traverse direction. A concave curved section 106 is positioned between the side section 108 and the top section 104.

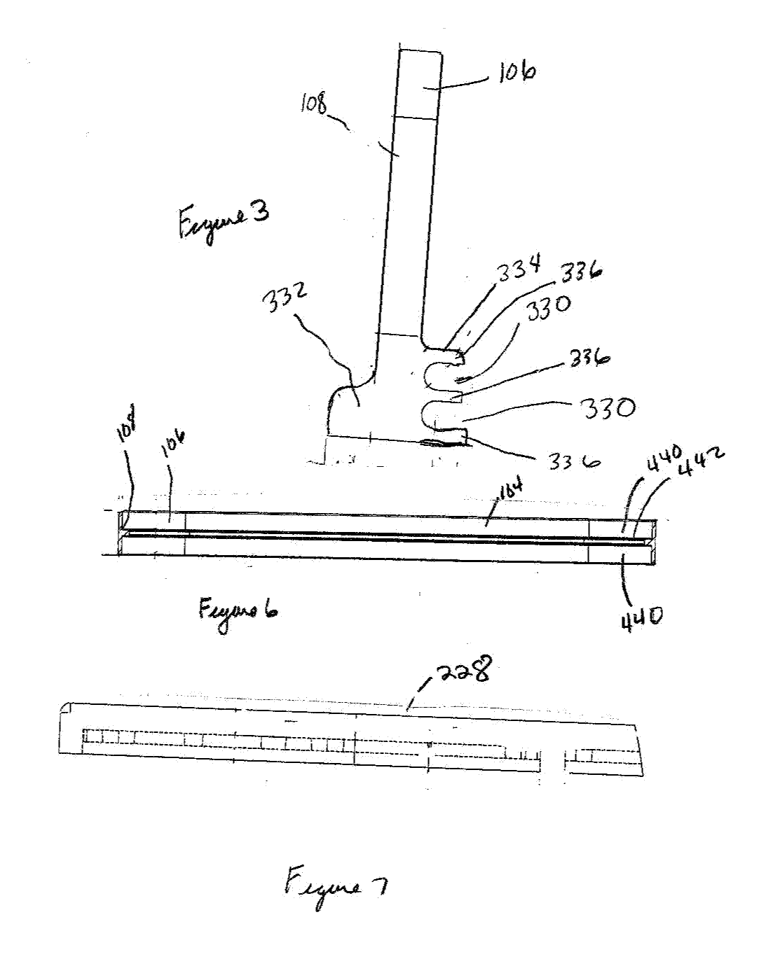

FIG. 2 illustrates a sectional view showing the exterior surface of the base section 102 which includes a first raised rib projection 222 which is formed at a first angle, a second raised rib projection 224 which is formed at a second angle being approximately 90° from the first raised rib projection 222 and a third raised rib projection 226 which extends across the base section 102. The base section 102 includes a front edge 252, a back edge 256 and a side edge 254. The back edge 256 is connected to the side edge 254 by a curved edge 258. The base section 102 includes a base extending portion 228 which extends in the distal direction to provide additional stability for the DrapeCaddy 100 and which does not necessarily extend across the entire width of the base section 102.

FIG. 3 illustrates a side view of the DrapeCaddy 100. The side section 108 includes a side extending portion 332 which extends in the distal direction to cooperate with the side edge 254 of the base section 102, and a substantially W shaped portion 332 which includes side arms 336 defined by apertures 330 to cooperate with an anesthesia circuit tubing (primarily), defibrillator cables, and assorted other lines to include IV tubing and ECG cables.

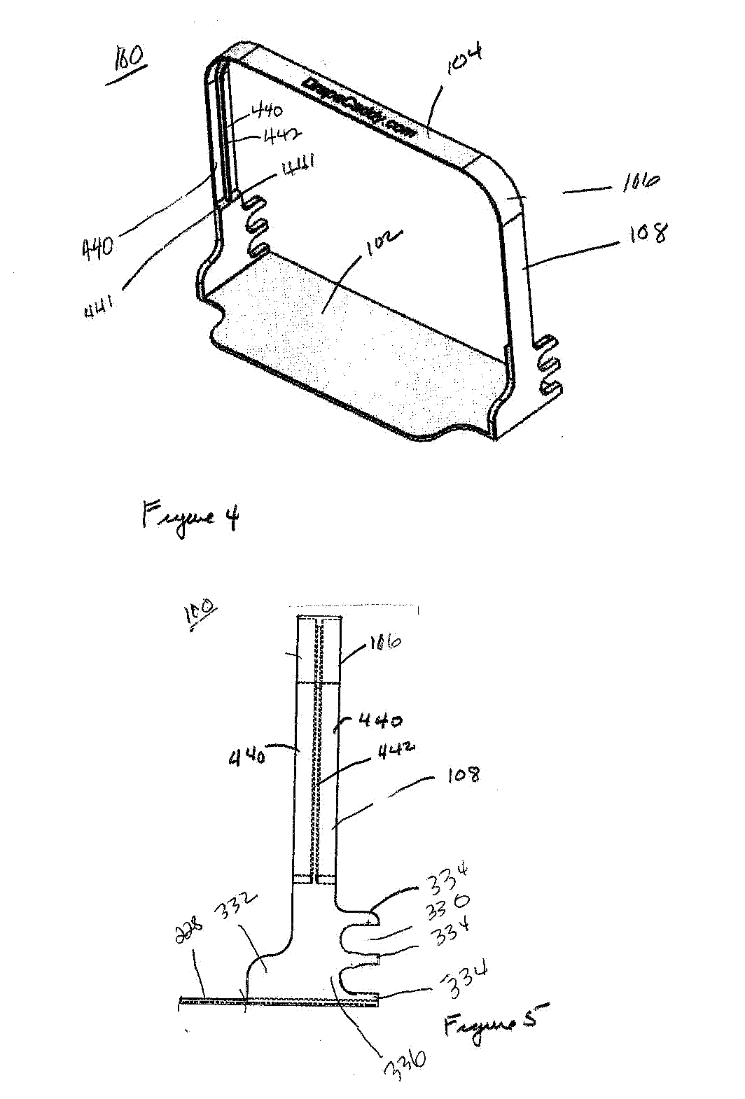

FIG. 4 illustrates a perspective view of the DrapeCaddy 100. The side section 108, the top section 104 and the concave curved section 106 includes a reduced thickness portion 440 which extends from the two edges of the side section 108, the top section 104 and the concave curved section 106 inwards to the inward extending projection 442. FIG. 4 additionally illustrates a chamfer 441 between the reduced thickness portion 440 and the W shaped portion 332.

FIG. 5 illustrates the cross-sectional view of the DrapeCaddy 100, and FIG. 5 illustrates the reduced thickness portion 440 and the inward extending projection 442 which extends inwards on the interior side edge of the side section 108, the curved concave section 106 and the top section 104.

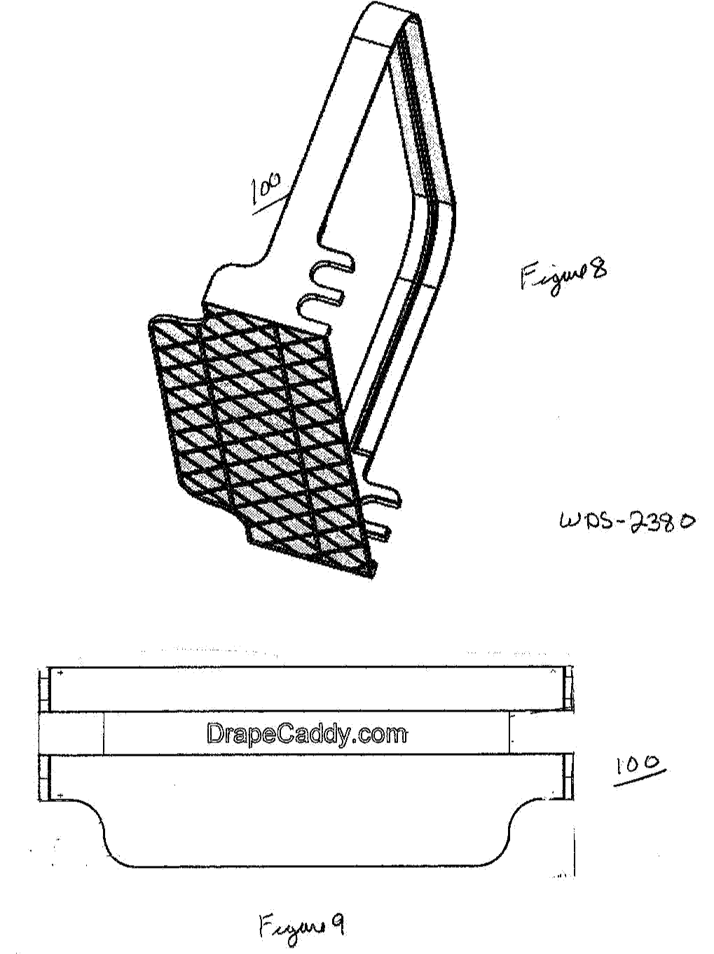

FIG. 6 illustrates cross section CC illustrated on FIG. 1. FIG. 6 shows the reduced thickness portion 440 and the inward extending projection 442.

FIG. 7 illustrates a side view of the extending portion 228.

FIG. 8 illustrates a perspective view of the DrapeCaddy 100.

FIG. 9 illustrates a top view of the DrapeCaddy 100.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1. A surgical drape support apparatus, comprising:
a base section;
a side section coupled to the base section;
a curved section coupled to the side section;
a top section coupled to the curved section.

2. A surgical drape support apparatus as in claim 1, wherein the side section includes a reduced thickness portion.

3. A surgical drape support apparatus as in claim 2, wherein the curved section includes the reduced thickness portion.

4. A surgical drape support apparatus as in claim 3, wherein the top section includes the reduced thickness portion.

5. A surgical drape support apparatus as in claim 1, wherein the side section includes an inward extending projection.

6. A surgical drape support apparatus as in claim 5, wherein the curved section includes the inward extending projection.

7. A surgical drape support apparatus as in claim 6, wherein the top section includes the inward extending projection.

8. A surgical drape support apparatus as in claim 1, wherein said base section includes a first raised rib projection.

9. A surgical drape support apparatus as in claim 8, wherein the base section includes a second raised rib projection substantially perpendicular to the first raised rib projection.

10. A surgical drape support apparatus as in claim 1, wherein the base section includes an extending portion.

11. A surgical drape support apparatus as in claim 1, wherein the side section includes a side extending portion.

12. A surgical drape support apparatus as in claim 1, wherein the side section includes a W shaped portion.
